(19) United States
(12) Patent Application Publication
(10) Pub. No.: US 2022/0155213 A1
(43) Pub. Date: May 19, 2022

(54) ERYTHROCYTE DIFFERENTIATION MONITORING APPARATUS AND ERYTHROCYTE DIFFERENTIATION MONITORING METHOD

(71) Applicant: OLYMPUS CORPORATION, Tokyo (JP)

(72) Inventors: Takuma DEZAWA, Tokyo (JP); Tomio ENDO, Tokyo (JP)

(73) Assignee: OLYMPUS CORPORATION, Tokyo (JP)

(21) Appl. No.: 17/591,006

(22) Filed: Feb. 2, 2022

Related U.S. Application Data
(63) Continuation of application No. PCT/JP2020/001798, filed on Jan. 21, 2020.

(51) Int. Cl. G01N 15/14; C12N 5/078; G01N 21/47

(52) U.S. Cl. CPC G01N 15/1456; G01N 2015/0073; G01N 21/47; C12N 5/0641

(57) ABSTRACT

An erythrocyte differentiation monitoring apparatus includes a laser light source that radiates a pulsed laser beam in a hemoglobin absorption wavelength range onto a cell within a culture container, a probe that receives a photoacoustic wave emitted from the cell within the culture container as a result of the cell being irradiated with the pulsed laser beam emitted from the laser light source, and a processor that evaluates the progress of differentiation of the cell into an erythrocyte based on the intensity of the photoacoustic wave received by the probe.

### CROSS-REFERENCE TO RELATED APPLICATIONS

This is a continuation of International Application PCT/JP2020/001798 which is hereby incorporated by reference herein in its entirety.

### TECHNICAL FIELD

The present invention relates to erythrocyte differentiation monitoring apparatuses and erythrocyte differentiation monitoring methods.

### BACKGROUND ART

In the field of regenerative medicine, in recent years, technology for producing blood cells from stem cells, such as iPS cells, is being established and has become a prospective solution for the lack of blood for transfusion. For example, there have been reports regarding a method for inducing differentiation from iPS cells into erythrocytes (e.g., see Non Patent Literature 1).

Producing an erythrocyte from a stem cell requires proper differentiation from a stem cell into an erythrocyte. Since it is wasteful to keep culturing a cell that does not differentiate properly, it is desirable to check whether the differentiation from a stem cell into an erythrocyte is being properly performed during the culture process. A known technique in the related art involves evaluating a colony of iPS cells based on phase-difference observation (e.g., see Patent Literature 1).

### CITATION LIST

### Non Patent Literature

### {NPL 1}

### Stem Cell Reports, Volume 1, Issue 6, p. 499

### Patent Literature

### {PTL 1}

PCT International Publication No. WO 2011/010449

### SUMMARY OF INVENTION

A first aspect of the present invention provides an erythrocyte differentiation monitoring apparatus including a laser light source that radiates a laser beam in a hemoglobin absorption wavelength range onto a cell within a culture container, an acoustic wave receiver that receives a photoacoustic wave emitted from the cell as a result of the cell being irradiated with the laser beam, and a processor that evaluates a progress of differentiation of the cell into an erythrocyte based on an intensity of the photoacoustic wave received by the acoustic wave receiver and outputs an evaluation result.

A second aspect of the present invention provides an erythrocyte differentiation monitoring method including radiating a laser beam in a hemoglobin absorption wavelength range onto a cell, receiving a photoacoustic wave emitted from the cell as a result of the cell being irradiated with the laser beam, and evaluating a progress of differentiation of the cell into an erythrocyte based on an intensity of the received photoacoustic wave and outputting an evaluation result.

### BRIEF DESCRIPTION OF DRAWINGS

FIG. 1 schematically illustrates the configuration of an erythrocyte differentiation monitoring apparatus according to a first embodiment of the present invention.

FIG. 2 illustrates an erythroid differentiation process.

FIG. 3 is a flowchart illustrating an erythrocyte differentiation monitoring method according to the first embodiment of the present invention.

FIG. 4 is a graph illustrating an example of a change in the amount of hemoglobin.

FIG. 5 is a flowchart illustrating an example where a temporal change in the intensity of a photoacoustic wave is acquired in accordance with the erythrocyte differentiation monitoring method according to the first embodiment of the present invention.

FIG. 6 illustrates an example of a photoacoustic image acquired by the erythrocyte differentiation monitoring apparatus in FIG. 1.

FIG. 7 schematically illustrates the configuration of an erythrocyte differentiation monitoring apparatus according to a modification of the first embodiment of the present invention.

FIG. 8 schematically illustrates the configuration of an erythrocyte differentiation monitoring apparatus according to a second embodiment of the present invention.

FIG. 9 illustrates an example of a photoacoustic image acquired by the erythrocyte differentiation monitoring apparatus in FIG. 8.

FIG. 10 illustrates an example of a cell image acquired by the erythrocyte differentiation monitoring apparatus in FIG. 8.

FIG. 11 illustrates an example of a superimposed image obtained by superimposing the photoacoustic image in FIG. 9 and the cell image in FIG. 10 on each other.

FIG. 12 is a flowchart illustrating an erythrocyte differentiation monitoring method according to the second embodiment of the present invention.

FIG. 13 schematically illustrates the configuration of an erythrocyte differentiation monitoring apparatus according to a modification of the second embodiment of the present invention.

FIG. 14 schematically illustrates the configuration of an erythrocyte differentiation monitoring apparatus according to a third embodiment of the present invention.

FIG. 15 schematically illustrates the configuration of an erythrocyte differentiation monitoring apparatus according to a modification of the third embodiment of the present invention.

FIG. 16 schematically illustrates the configuration of a culture container and an agitating mechanism in an erythrocyte differentiation monitoring apparatus according to a fourth embodiment of the present invention.

FIG. 17 schematically illustrates the configuration of the erythrocyte differentiation monitoring apparatus according to the fourth embodiment of the present invention, as viewed from above.

FIG. 18 is a flowchart illustrating an example where a temporal change in the intensity of a photoacoustic wave is acquired in accordance with an erythrocyte differentiation monitoring method according to the fourth embodiment of the present invention.

FIG. 19 is a vertical sectional view of a culture container and a stereo measurement device used in the erythrocyte differentiation monitoring apparatus in FIG. 17.

FIG. 20 is a cross-sectional view schematically illustrating the configuration of the stereo measurement device in FIG. 19.

FIG. 21 schematically illustrates a part of an erythrocyte differentiation monitoring apparatus provided with an additional flow path outside the culture container.

### DESCRIPTION OF EMBODIMENTS

### First Embodiment

An erythrocyte differentiation monitoring apparatus and an erythrocyte differentiation monitoring method according to a first embodiment of the present invention will be described below with reference to the drawings.

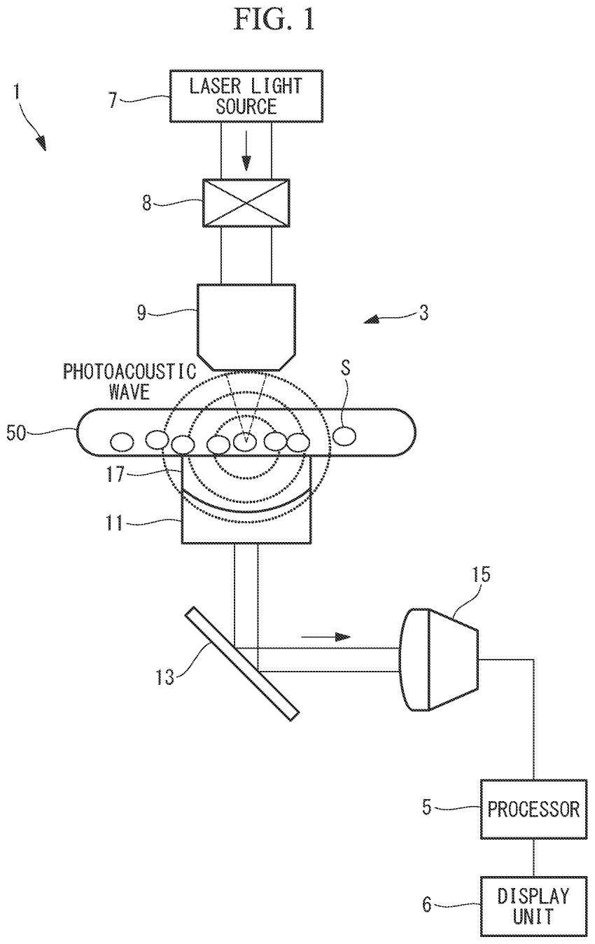

As shown in FIG. 1, an erythrocyte differentiation monitoring apparatus 1 according to this embodiment includes a photoacoustic optical system 3 and a processor 5 that evaluates the progress of differentiation of a cell S into an erythrocyte. Furthermore, the erythrocyte differentiation monitoring apparatus 1 is connected to a display unit 6 of, for example, a monitor or a terminal.

The photoacoustic optical system 3 includes a laser light source 7 that emits a pulsed laser beam, an objective lens 9 that focuses the pulsed laser beam emitted from the laser light source 7 onto the cell S within a culture container (container) 50, an acoustic lens 11 that converts a photoacoustic wave emitted from the cell S into a collimated wave, an acoustic-wave reflection member 13 that reflects the photoacoustic wave converted into the collimated wave by the acoustic lens 11, and a probe (acoustic wave receiver) 15 that receives the photoacoustic wave reflected by the acoustic-wave reflection member 13. The photoacoustic optical system 3 further includes a scanning unit 8 that scans the pulsed laser beam emitted from the laser light source 7.

The probe (acoustic wave receiver) 15 is, for example, an ultrasonic vibrator array having an array of ultrasonic vibrators. Each ultrasonic vibrator is formed of a piezoelectric element composed of a piezoelectric ceramic material or a polymer film, such as a polyvinylidene fluoride film. When receiving a photoacoustic wave, each ultrasonic vibrator has a function for converting the reception signal of the photoacoustic wave into an electrical signal indicating the intensity of the photoacoustic wave.

A pulsed laser beam can be instantaneously increased in energy density. A pulsed laser beam is superior to a laser beam emitted from a continuous-wave laser light source since a photoacoustic wave can be efficiently generated while optical damage to the cell S can be suppressed due to a short time period in which the laser beam is radiated onto the cell S. However, the laser light source 7 that emits a pulsed laser beam does not necessarily have to be used. An inexpensive laser light source that emits a continuous-wave (CW) laser beam may be used as an alternative, although such a laser light source has lower efficiency for generating a photoacoustic wave.

The laser light source 7 generates a pulsed laser beam in a hemoglobin absorption wavelength range. For example, the laser light source 7 desirably generates any one of or a plurality of a pulsed laser beam with a wavelength of 555 nm as a peak absorption wavelength for hemoglobin (Hb), pulsed laser beams with wavelengths of 541 nm and 576 nm as peak absorption wavelengths for oxygenated hemoglobin (HbO₂), and a pulsed laser beam with a wavelength near 1000 nm as a near-infrared wavelength. Because the intensity of a photoacoustic wave increases in accordance with the amount of light absorbed by a molecule, a photoacoustic wave can be efficiently acquired by using a pulsed laser beam with a peak absorption wavelength for each molecule.

The wavelength to be used is not particularly limited so long as the wavelength is absorbed by hemoglobin or oxygenated hemoglobin. The near-infrared wavelength is not particularly limited so long as it does not overlap with the absorption range of phenol red serving as a medium component. In addition, the wavelength at the isosbestic point of hemoglobin or oxygenated hemoglobin may be used. Hemoglobin may sometimes change into oxygenated hemoglobin by being affected by the amount of ambient oxygen (oxygen partial pressure). This change is reversible, meaning that oxygenated hemoglobin may sometimes change into hemoglobin. In this case, since hemoglobin and oxygenated hemoglobin absorb the same amount of light, the effect of the change in the content ratio of hemoglobin and oxygenated hemoglobin due to the effect of the oxygen partial pressure can be negated. A pulsed laser beam generated by the laser light source 7 will simply be referred to as “laser beam” hereinafter.

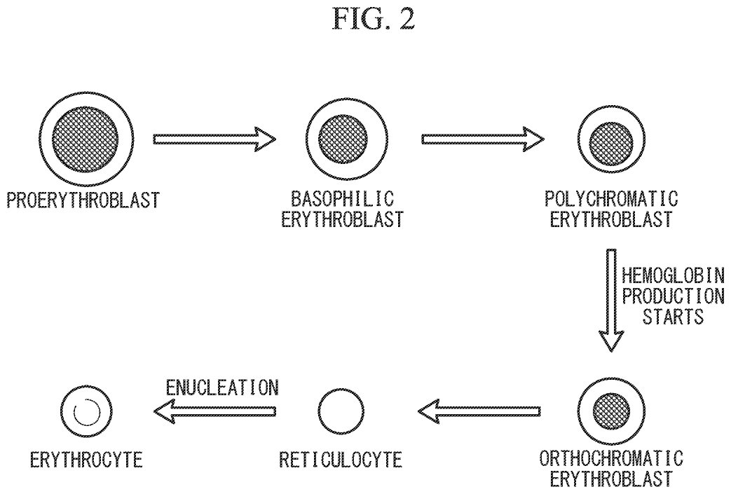

In the process of differentiation from a stem cell into an erythrocyte, for example, production of hemoglobin starts when the stem cell differentiates into an orthochromatic erythroblast, as shown in FIG. 2. When a laser beam in the hemoglobin absorption wavelength range is radiated onto the cell S, if the cell S has differentiated into an orthochromatic erythroblast, the laser beam is absorbed by the hemoglobin in the cell S. Then, thermal expansion occurs instantaneously in the hemoglobin having absorbed the laser beam, so that a photoacoustic wave (photoacoustic effect) is emitted from the cell S. On the other hand, if the cell S has not properly differentiated into, for example, an orthochromatic erythroblast, or is still in an undifferentiated state, the laser beam is not absorbed since hemoglobin is not produced in the cell S, so that a photoacoustic wave is not emitted from the cell S.

The culture container 50 is, for example, a flask or a dish.

The acoustic lens 11 is composed of, for example, SiO₂ or sapphire. The acoustic lens 11 converts a photoacoustic wave into a collimated wave, so that the sound collecting efficiency of the probe 15 can be enhanced. The acoustic lens 11 may have a solid or liquid transmission member 17 that transmits a photoacoustic wave and that is set in close contact with the culture container 50 by being interposed between the acoustic lens 11 and the base of the culture container 50.

The acoustic-wave reflection member 13 is formed of an optical component, such as a prism coated with a high acoustic-impedance material. An example of a high acoustic-impedance material is silicone oil. The acoustic-wave reflection member 13 transmits light and reflects a photoacoustic wave transmitted through the acoustic lens 11 toward the probe 15.

A liquid photoacoustic transmission medium (not shown), such as water, transmitting a photoacoustic wave fills the space from the objective lens 9 to the culture container 50, the acoustic lens 11, the acoustic-wave reflection member 13, and the probe 15. The probe 15 may receive a photoacoustic wave from the cell S in a state where the probe 15 is set in close contact with the base of the culture container 50, such as a flask or a dish.

The scanning unit 8 is, for example, a galvanometer mirror or MEMS mirror containing a driving source (e.g., motor). The scanning unit 8 is controlled by the processor 5 and scans a laser beam emitted from the laser light source 7 two-dimensionally within the culture container 50. The scanning unit 8 may further be configured to scan the laser beam in an optical-axis direction of the objective lens 9.

The processor 5 may include hardware. The hardware may include at least one of a circuit that processes a digital signal and a circuit that processes an analog signal. The processor 5 may include, for example, one or more circuit devices, such as ICs (integrated circuits), on a circuit board, or one or more circuit elements, such as resistors and capacitors.

The processor 5 may be a CPU (central processing unit). The processor 5 used may be of various types including a GPU (graphics processing unit) and a DSP (digital signal processor). The processor 5 used may be a hardware circuit equipped with an ASIC (application specific integrated circuit) or an FPGA (field-programmable gate array). The processor 5 may include, for example, a filter circuit and an amplification circuit for processing an analog signal.

For example, the processor 5 realizes the following process in accordance with a program stored in a memory (not shown). For example, the processor 5 evaluates the progress of differentiation of the cell S into an erythrocyte based on the intensity, that is, the amplitude, of a photoacoustic wave received by the probe 15. Furthermore, the processor 5 calculates the amount of hemoglobin produced in the cell S based on the intensity of the photoacoustic wave received by the probe 15. Moreover, the processor 5 estimates the cell type of the cell S from the calculated amount of produced hemoglobin.

The processor 5 transmits the evaluation result, the calculated amount of produced hemoglobin, and the estimated cell type of the cell S to the display unit 6. A specific output destination for the evaluation result, the calculation result, and the estimation result obtained by the processor 5 is not particularly limited so long as the output destination is a terminal equipped with a display unit 6, and may be, for example, a notebook computer, a desktop computer, a smartphone, or a tablet terminal.

For example, the memory may be a freely-chosen semiconductor memory, such as a RAM (random access memory). When the stored program is to be executed, the memory functions as a working memory that stores a program or data stored in a nonvolatile memory, such as a hard disk or a flash memory.

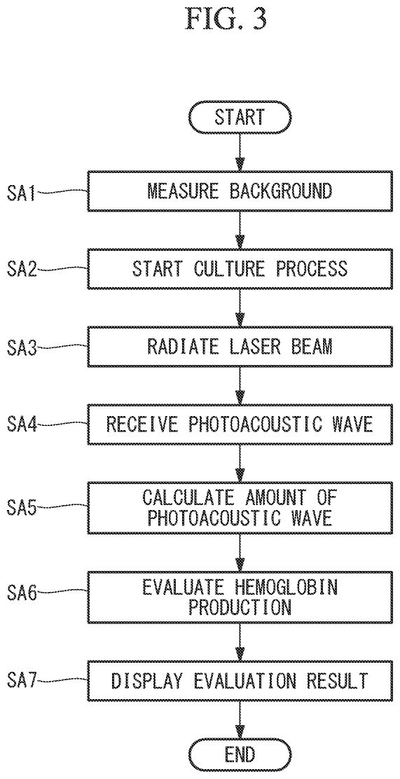

As shown in a flowchart in FIG. 3, the erythrocyte differentiation monitoring method according to this embodiment includes radiating a laser beam in the hemoglobin absorption wavelength range emitted from the laser light source 7 onto the cell S (step SA3), using the probe 15 to receive a photoacoustic wave emitted from the cell S as a result of the cell S being irradiated with the laser beam (step SA4), evaluating the progress of differentiation of the cell S into an erythrocyte based on the intensity of the photoacoustic wave received by the probe 15 (step SA6), and outputting the evaluation result.

The operation of the erythrocyte differentiation monitoring apparatus 1 having the above-described configuration and the erythrocyte differentiation monitoring method will now be described.

In a case where a cell S is to be monitored by using the erythrocyte differentiation monitoring apparatus 1 and the erythrocyte differentiation monitoring method according to this embodiment, the background is first measured (step SA1) to eliminate the effect of absorption of a laser beam by a medium component and other components.

In detail, before seeding the cell S in the culture container 50, a laser beam in the hemoglobin absorption wavelength range, such as a laser beam with a wavelength of 555 nm, is scanned over a culture medium (not shown), such as phenol red, contained together with the cell S in the culture container 50. Accordingly, a photoacoustic signal emitted from the medium component and other components is acquired by the probe 15. The photoacoustic signal acquired by the probe 15 is transmitted as a background signal to the processor 5.

Subsequently, the cell S is seeded in the culture container 50 containing the culture medium, so that the culture process starts (step SA2). In the case of a plane culture process, the culture container 50 having the cell S seeded therein is accommodated in an incubator (not shown), such that the cell S is cultured within the incubator.

Then, observation of the cell S begins, and a laser beam with a wavelength of 555 nm belonging to the hemoglobin absorption wavelength range is generated from the laser light source 7. The laser beam emitted from the laser light source 7 is scanned by the scanning unit 8 and is subsequently radiated onto the cell S within the culture container 50 by the objective lens 9 (step SA3). If a temporal change in the intensity of the photoacoustic wave is to be monitored, the scanning unit 8 repeatedly scans the laser beam. Alternatively, the lens may be moved in a scanning fashion together with the scanning process.

If hemoglobin is produced within the cell S, a photoacoustic wave is emitted from the cell S as a result of the cell S being irradiated with the laser beam in the hemoglobin absorption wavelength range. If hemoglobin is not produced within the cell S, a photoacoustic wave is not emitted from the cell S even if the cell S is irradiated with the laser beam in the hemoglobin absorption wavelength range. It may be confirmed whether a photoacoustic wave has been appropriately acquired by scanning the laser beam. If the cell S is appropriately irradiated with the laser beam, scanning the laser beam should cause the focus position to deviate from the cell S, thus causing attenuation of the intensity of the photoacoustic wave.

When a photoacoustic wave is emitted from the cell S as a result of the cell S being irradiated with the laser beam in the hemoglobin absorption wavelength range, the photoacoustic wave travels through the transmission member 17 and is converted into a collimated wave by the acoustic lens 11. Then, the collimated photoacoustic wave transmitted through the acoustic lens 11 is reflected by the acoustic-wave reflection member 13 so as to be received by the probe 15 (step SA4). A photoacoustic signal acquired by the probe 15 is transmitted to the processor 5.

Subsequently, the processor 5 subtracts the background signal from the photoacoustic signal transmitted from the probe 15, whereby the intensity of the photoacoustic wave from the cell S is calculated (step SA5).

If the intensity of the photoacoustic wave from the cell S is a positive value, the processor 5 evaluates that the production of hemoglobin has started in the cell S irradiated with the laser beam, that is, the differentiation of the cell S is properly progressing (step SA6). Moreover, the processor 5 calculates the amount of hemoglobin produced in the cell S and estimates the cell type. In contrast, if the intensity of the photoacoustic wave calculated in step SA5 is near zero or is a negative value, the processor 5 evaluates that hemoglobin is not produced in the cell S irradiated with the laser beam, that is, the differentiation of the cell S is not properly progressing.

The evaluation result obtained by the processor 5, the amount of hemoglobin produced in the cell S calculated by the processor 5, and the estimated cell type of the cell S are transmitted to the display unit 6, and are displayed by the display unit 6 (step SA7).

As described above, in the erythrocyte differentiation monitoring apparatus 1 and the erythrocyte differentiation monitoring method according to this embodiment, the processor 5 determines whether or not differentiation from a stem cell into an erythrocyte is properly progressing based on the intensity of a photoacoustic wave received by the probe 15. Accordingly, the differentiation from a stem cell into an erythrocyte can be quantitatively monitored in a simple and accurate manner, as compared with a case where the differentiation of the cell S is confirmed visually.

The larger the amount of hemoglobin in the cell S, the larger the amount of absorption of the laser beam in the hemoglobin absorption wavelength range and the higher the intensity of the photoacoustic wave emitted from the cell S. In other words, the amount of hemoglobin produced in the cell S is proportional to the intensity of the photoacoustic wave received by the probe 15. Therefore, the processor 5 can readily and quantitatively calculate the amount of hemoglobin produced in the cell S based on the calculated intensity of the photoacoustic wave from the cell S.

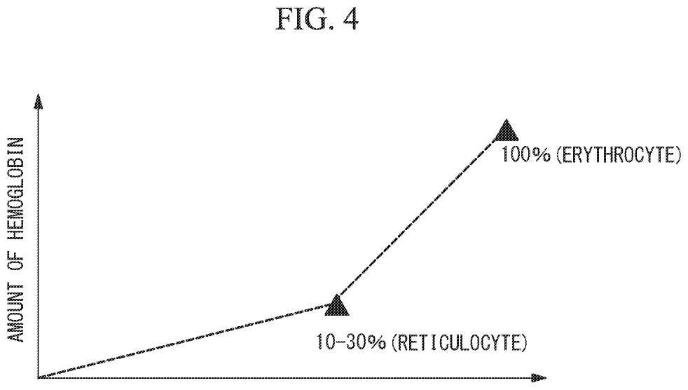

Furthermore, for example, as shown in FIG. 4, in the process of differentiation from a stem cell into an erythrocyte, the amount of produced hemoglobin increases gradually from an orthochromatic erythroblast to a reticulocyte. On the other hand, the amount of produced hemoglobin increases rapidly from a reticulocyte to an erythrocyte. Therefore, the processor 5 can readily estimate the cell type based on the calculated amount of produced hemoglobin. In FIG. 4, the ordinate axis denotes the amount of hemoglobin in the cell S, whereas the abscissa axis denotes the cell type.

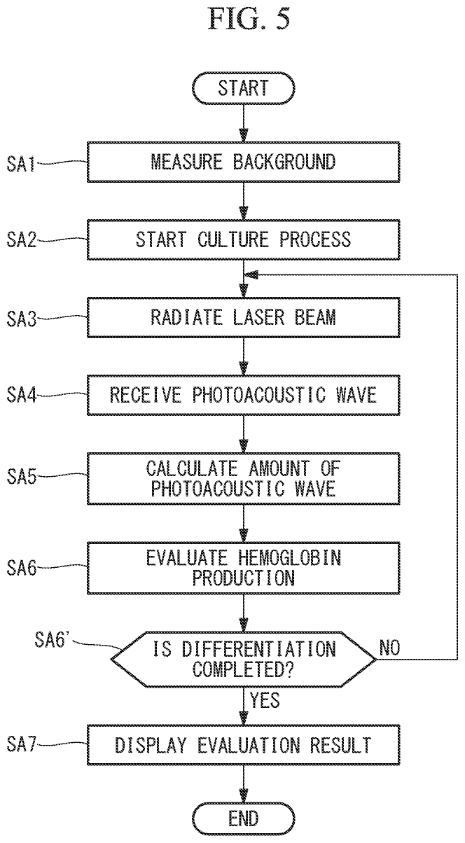

In this embodiment, it is desirable that a temporal change in the intensity of the photoacoustic wave from the cell S be acquired and be monitored until the differentiation is completed. An example where a temporal change in the intensity of the photoacoustic wave is acquired will be described below with reference to a flowchart in FIG. 5.

If the processor 5 evaluates that the differentiation of the cell S is properly progressing, the processor 5 further determines whether or not the differentiation of the cell S is completed (step SA6′).

The processor 5 may determine that the differentiation is completed when the detected intensity of the photoacoustic wave exceeds a predetermined threshold value. Furthermore, the processor 5 may monitor a temporal change in the intensity of the photoacoustic wave and determine that the differentiation is completed when the rate of change in the intensity has peaked out. The evaluation result obtained by the processor 5, the calculated amount of produced hemoglobin, and the estimated cell type may individually be displayed immediately by the display unit 6 or may collectively be displayed by the display unit 6 (step SA7). If the processor 5 determines that the differentiation is not completed, the processor 5 may control the laser light source 7 to radiate a laser beam again.

It is desirable that the temporal change in the intensity of the photoacoustic wave be displayed on a graph.

A user can acquire the intensity of the photoacoustic wave emitted from the cell S to determine whether or not differentiation into an erythrocyte has been performed. Furthermore, by acquiring the temporal change in the intensity of the photoacoustic wave, it is possible to evaluate the degree of progress of the differentiation, the level of differentiation efficiency within the culture container 50, and the differentiation efficiency compared with that in a past culture process. Accordingly, the culture process can be performed quantitatively and efficiently. If the differentiation efficiency is low, it is possible to decide whether to stop the culture process in mid-course, thereby eliminating wasted time and reducing costs.

Since this embodiment relates to a plane culture process, the processor 5 may generate a photoacoustic image based on positional coordinates from where the photoacoustic wave is emitted and the intensity of the photoacoustic wave. Moreover, the generated photoacoustic image may be displayed on the display unit 6. The positional coordinates from where the photoacoustic wave is emitted can be determined from the scan position of the laser beam.

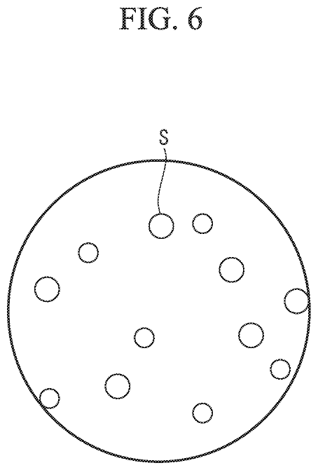

For example, as shown in FIG. 6, the photoacoustic image may indicate the positional coordinates from where the photoacoustic wave is emitted, that is, the location where hemoglobin is produced in the culture container 50, by using a graphic symbol, such as a circle or a square. Furthermore, the color density and the size of the graphic symbol indicating the location where hemoglobin is produced may be varied in accordance with the amount of produced hemoglobin. The positional coordinates from where the photoacoustic wave is emitted may be calculated by the processor 5 based on the irradiation position of the laser beam.

According to this modification, it is possible not only to determine whether hemoglobin is produced in the cell S, but also to ascertain at a glance how much the differentiation of the cell S has progressed within the culture container 50.

As an alternative to this embodiment in which the container is described as being the culture container 50, such as a flask or a dish, the container used may be a soft culture bag composed of, for example, vinyl. In the case where a culture bag is used as the culture container 50, since the probe 15 can be readily set in close contact with the culture bag, a photoacoustic wave may be acquired in a state where the probe 15 is in close contact with any area of the culture bag. With the increased contact between the culture container 50 and the probe 15, the transmissibility of the photoacoustic wave increases, so that the photoacoustic wave can be acquired more efficiently.

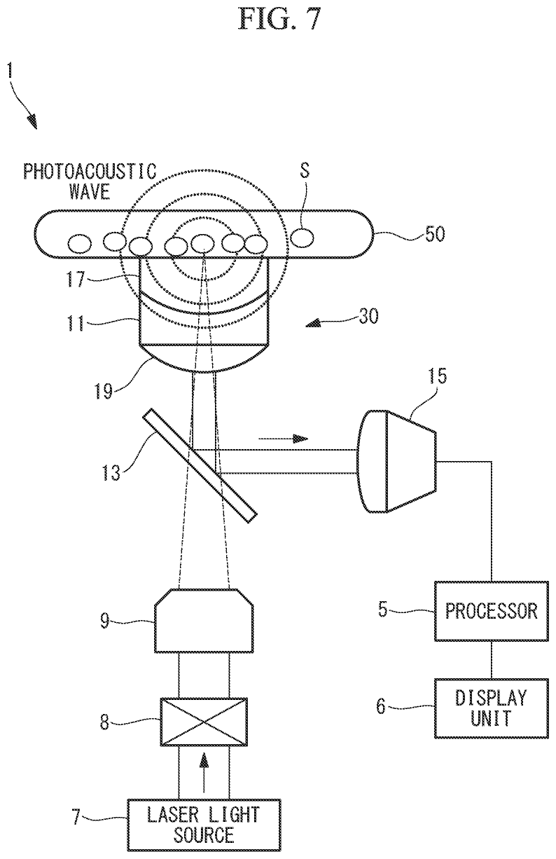

In this embodiment, the erythrocyte differentiation monitoring apparatus 1 includes the photoacoustic optical system 3 that radiates a pulsed laser beam from above the cell S. Alternatively, for example, as shown in FIG. 7, the erythrocyte differentiation monitoring apparatus 1 may include a photoacoustic optical system 30 that radiates a pulsed laser beam from below the cell S. The photoacoustic optical system 30 includes the laser light source 7, the scanning unit 8, the objective lens 9, the acoustic lens 11, the acoustic-wave reflection member 13, and the probe 15.

In the photoacoustic optical system 30, a laser beam emitted from the laser light source 7 is focused by the objective lens 9 via the scanning unit 8, and is subsequently transmitted through the acoustic-wave reflection member 13. Then, the laser beam transmitted through the acoustic-wave reflection member 13 travels through the acoustic lens 11 and the transmission member 17 and is radiated onto the cell S within the culture container 50. Subsequently, a photoacoustic wave emitted from the cell S travels through the transmission member 17 and is converted into a collimated wave by the acoustic lens 11, and is then reflected by the acoustic-wave reflection member 13 so as to be received by the probe 15. In the case in FIG. 7, the acoustic-wave reflection member 13 reflects an acoustic wave and transmits a laser beam.

In the photoacoustic optical system 30, a correction lens 19 may be disposed in close contact with the acoustic-wave-reflection-member-13-side surface of the acoustic lens 11, such that aberration of light occurring due to the acoustic lens 11 and the transmission member 17 may be corrected by the correction lens 19.

### Second Embodiment

Next, an erythrocyte differentiation monitoring apparatus and an erythrocyte differentiation monitoring method according to a second embodiment of the present invention will be described.

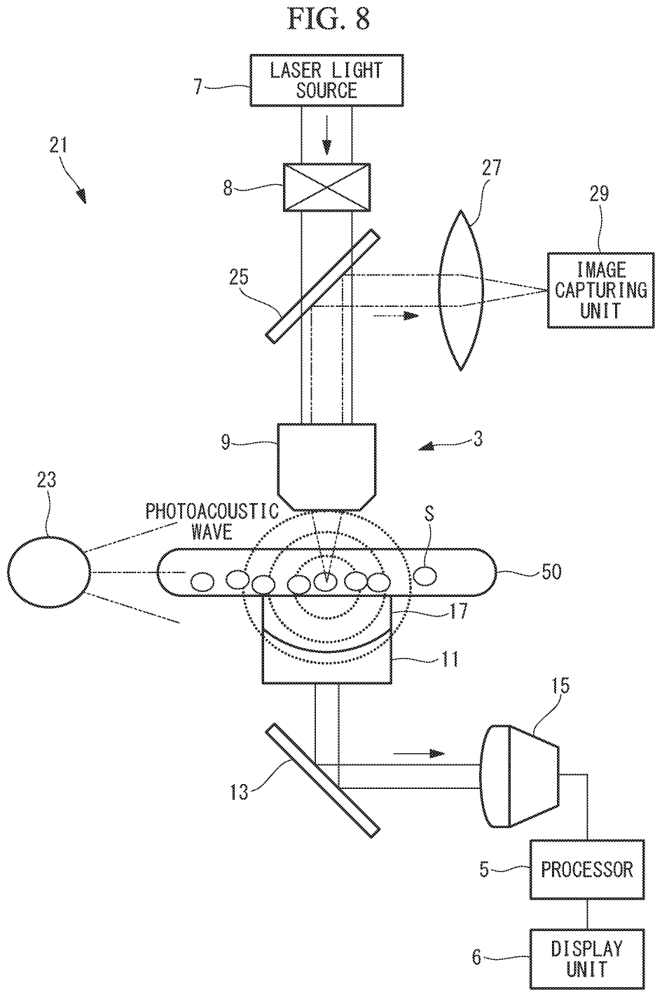

As shown in FIG. 8, an erythrocyte differentiation monitoring apparatus 21 according to this embodiment is different from that in the first embodiment in that the erythrocyte differentiation monitoring apparatus 21 includes a generic optical microscope that acquires a two-dimensional image of the cell S, in addition to the photoacoustic optical system 3. In other words, in contrast to the first embodiment in which only a photoacoustic wave is acquired, the erythrocyte differentiation monitoring apparatus 21 according to this embodiment acquires a cell image in addition to a photoacoustic wave.

In the following description, sections identical to those in the erythrocyte differentiation monitoring apparatus 1 and the erythrocyte differentiation monitoring method according to the first embodiment will be given the same reference signs, and descriptions thereof will be omitted.

The erythrocyte differentiation monitoring apparatus 21 includes an illumination light source 23 that radiates illumination light onto the cell S, a dichroic mirror 25 that reflects observation light emitted from the cell S, as a result of the cell S being irradiated with the illumination light, and collected by the objective lens 9, an imaging optical system 27 that forms an image of the observation light reflected by the dichroic mirror 25, and an image capturing unit 29 that captures the image formed by the imaging optical system 27. For example, the dichroic mirror 25 has the property of transmitting a laser beam with a wavelength ranging between 540 and 580 nm and reflecting other wavelength ranges.

The objective lens 9 radiates a laser beam from the laser light source 7 onto the cell S, and collects scattered light (observation light) emitted from the cell S irradiated with the illumination light from the illumination light source 23.

The dichroic mirror 25 reflects the scattered light collected by the objective lens 9 toward the image capturing unit 29, and transmits the laser beam from the laser light source 7 toward the objective lens 9.

The illumination light source 23 is, for example, an LED or a halogen lamp. For example, the illumination light source 23 is disposed alongside the culture container 50 and radiates the illumination light onto the culture container 50 from a direction intersecting the optical axis of the objective lens 9 without causing the illumination light to travel through the acoustic lens 11, thereby obliquely illuminating the cell S.

The image capturing unit 29 includes an image capturing element, such as a CCD or a CMOS element, and acquires image information of the cell S by capturing an image of the scattered light from the cell S. The image information of the cell S acquired by the image capturing unit 29 is transmitted to the processor 5.

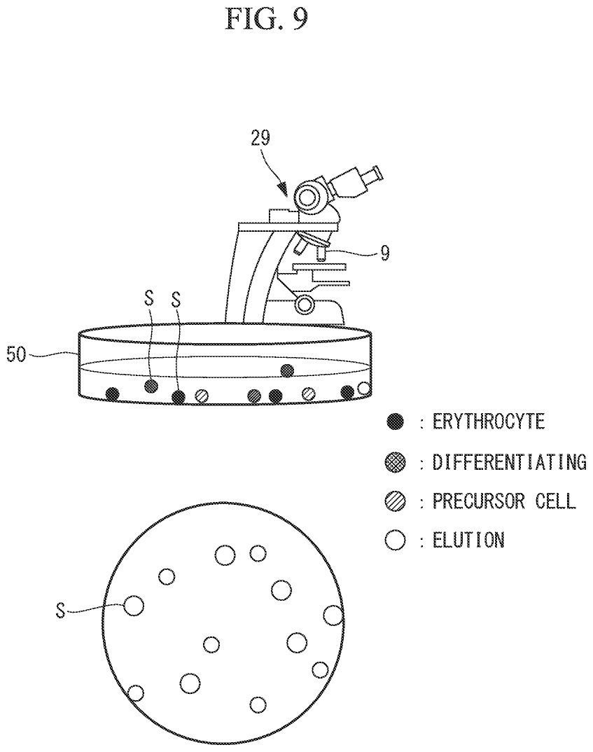

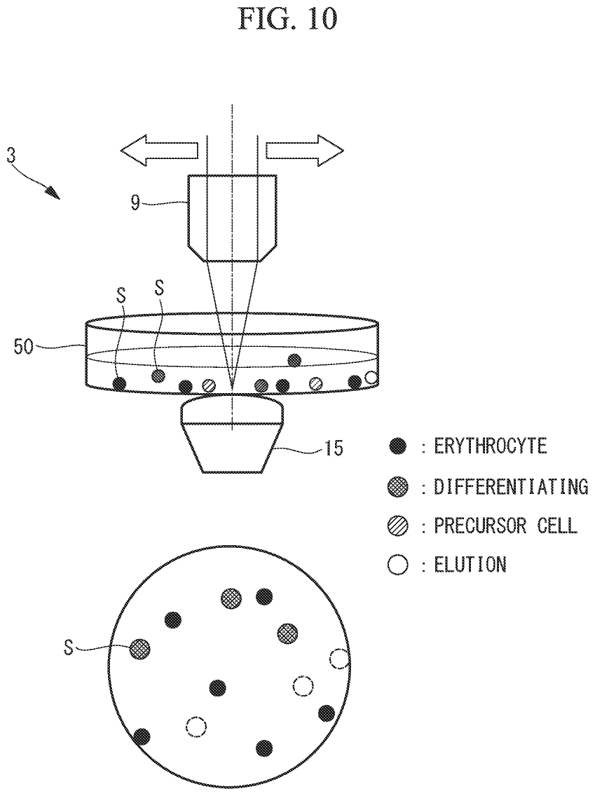

The processor 5 generates a photoacoustic image, as shown in FIG. 9, based on a photoacoustic wave of the cell S received by the probe 15. Moreover, the processor 5 generates a cell image, as shown in FIG. 10, based on the image information of the cell S transmitted from the image capturing unit 29.

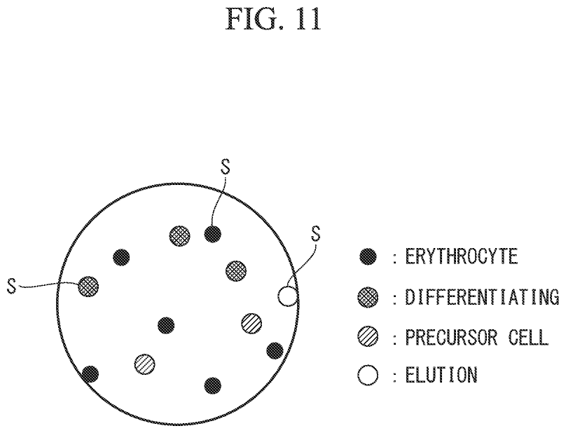

The processor 5 superimposes the generated photoacoustic image and cell image of the cell S to generate a superimposed image, as shown in FIG. 11, thereby determining the state of the cell S. The superimposed image generated by the processor 5 is displayed by the display unit 6.

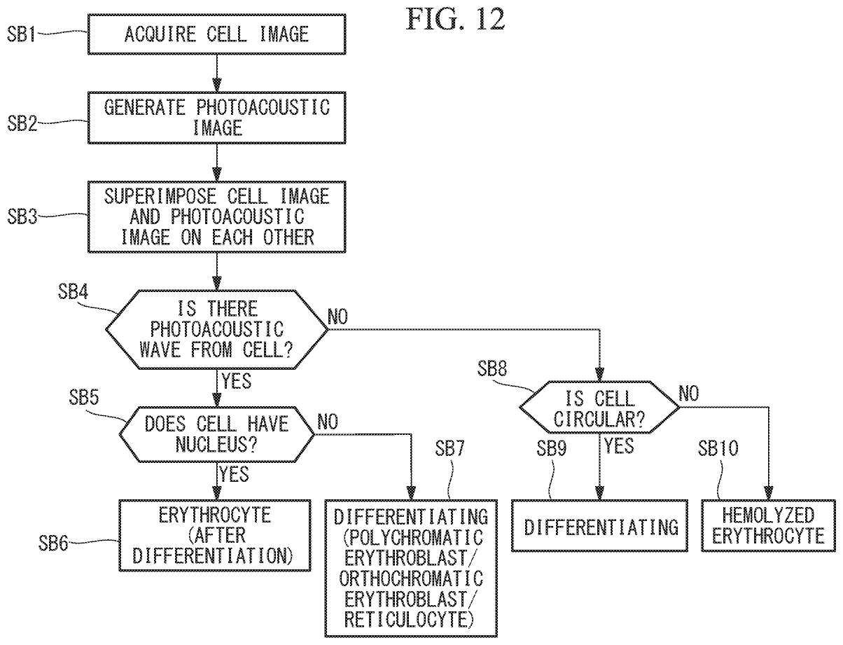

The operation of the erythrocyte differentiation monitoring apparatus 21 having the above-described configuration and the erythrocyte differentiation monitoring method will now be described with reference to a flowchart in FIG. 12.

In a case where a cell S is to be monitored by using the erythrocyte differentiation monitoring apparatus 21 according to this embodiment, the illumination light source 23 radiates illumination light onto the cell S within the culture container 50, and the image capturing unit 29 captures an image of scattered light from the cell S. Accordingly, the processor 5 generates a cell image, as shown in FIG. 9 (step SB1).

Subsequently, the laser light source 7 generates a laser beam in the hemoglobin absorption wavelength range, and radiates the laser beam onto the cell S via the scanning unit 8, the dichroic mirror 25, and the objective lens 9. A photoacoustic wave emitted from the cell S is transmitted through the transmission member 17 and the acoustic lens 11, and is subsequently reflected by the acoustic-wave reflection member 13 and received by the probe 15. Accordingly, the processor 5 generates a photoacoustic image, as shown in FIG. 10 (step SB2).

Subsequently, the processor 5 superimposes the generated cell image and photoacoustic image on each other, so as to generate a superimposed image, as shown in FIG. 11 (step SB3). Then, the processor 5 determines whether or not a photoacoustic wave is emitted from the cell S (step SB4). If it is determined that a photoacoustic wave is emitted from the cell S, the processor 5 performs image-processing on the superimposed image, and determines whether or not there is a nucleus in the cell S (step SB5).

If it is determined that there is no nucleus in the cell S, the processor 5 determines that the cell S has differentiated into an erythrocyte (step SB6). This is due to a so-called enucleation phenomenon where the cell nucleus vanishes during the differentiation stage into an erythrocyte. In contrast, if it is determined that there is a nucleus in the cell S, the processor 5 determines that the cell S is still undergoing the differentiation process, that is, the cell S has not become an erythrocyte yet (step SB7). In step SB7, the processor 5 may estimate whether the cell type is any one of a polychromatic erythroblast, an orthochromatic erythroblast, and a reticulocyte based on the intensity of the photoacoustic wave.

If it is determined in step SB4 that a photoacoustic wave is not emitted from the cell S, the processor 5 performs image-processing on the superimposed image, and determines whether or not the cell S has a circular shape (step SB8). If it is determined that the cell S is circular, the processor 5 determines that the cell S is undergoing the differentiation process, that is, the cell S is a proerythroblast or a basophilic erythroblast (step SB9). In contrast, if it is determined that the cell S is not circular, the processor 5 determines that the cell S is a hemolyzed erythrocyte (step SB10). The term “hemolyzed” refers to a state where the erythrocyte is broken due to a certain reason and is not able to maintain its blood cell shape, such as a circular shape or a spherical shape. Because the erythrocyte is broken, the hemoglobin cannot be maintained inside the cell, and the photoacoustic wave is not observed regardless of the fact that the differentiation has progressed.

Accordingly, in the erythrocyte differentiation monitoring apparatus 21 and the erythrocyte differentiation monitoring method according to this embodiment, a photoacoustic image and a cell image of the cell S can both be acquired. Therefore, it is not necessary to move the culture container 50 between a case where a photoacoustic image of the same cell S is to be acquired and a case where a cell image thereof is to be acquired. Thus, the same cell S can be readily and accurately associated between the photoacoustic image and the cell image, whereby the state of the cell S can be accurately determined. Furthermore, by obliquely illuminating the cell S, a three-dimensional cell image of a transparent and colorless cell S can be acquired.

In this embodiment, the illumination light source 23 may obliquely illuminate the cell S without causing the illumination light to travel through the acoustic lens 11, so that a photoacoustic image and a cell image may be acquired at the same time.

In this embodiment, based on a photoacoustic image and a cell image of a plurality of cells S included in the irradiation region of the laser beam and the illumination light within the culture container 50, the processor 5 may calculate the percentage of cells S differentiated into erythrocytes among the plurality of cells S within the region.

In this embodiment, a half mirror may be used in place of the dichroic mirror 25. Furthermore, in this embodiment, the laser light source 7 may be positionally interchanged with the imaging optical system 27 and the image capturing unit 29, such that the dichroic mirror 25 or the half mirror may reflect the laser beam from the laser light source 7 toward the objective lens 9 and transmit the scattered light from the objective lens 9 toward the imaging optical system 27 and the image capturing unit 29.

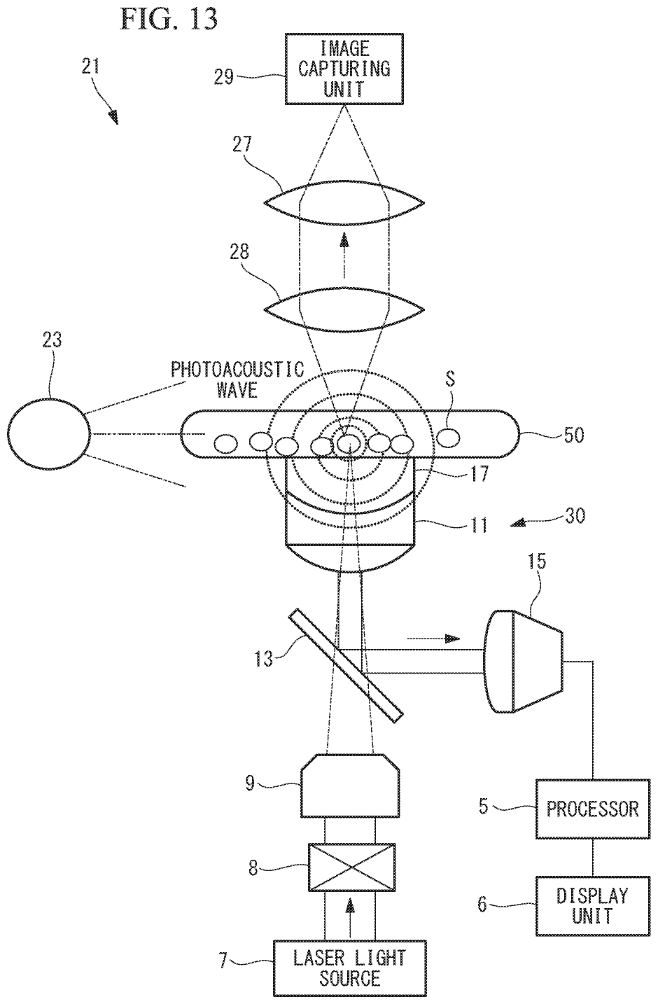

As an alternative to this embodiment in which the erythrocyte differentiation monitoring apparatus 21 includes the photoacoustic optical system 3, for example, the erythrocyte differentiation monitoring apparatus 21 may include the photoacoustic optical system 30, as shown in FIG. 13. In this case, the dichroic mirror 25 is not necessary. With this configuration, advantages similar to those in this embodiment can be achieved. In FIG. 13, reference sign 28 denotes a collecting lens that collects scattered light from the cell S.

### Third Embodiment

Next, an erythrocyte differentiation monitoring apparatus and an erythrocyte differentiation monitoring method according to a third embodiment of the present invention will be described.

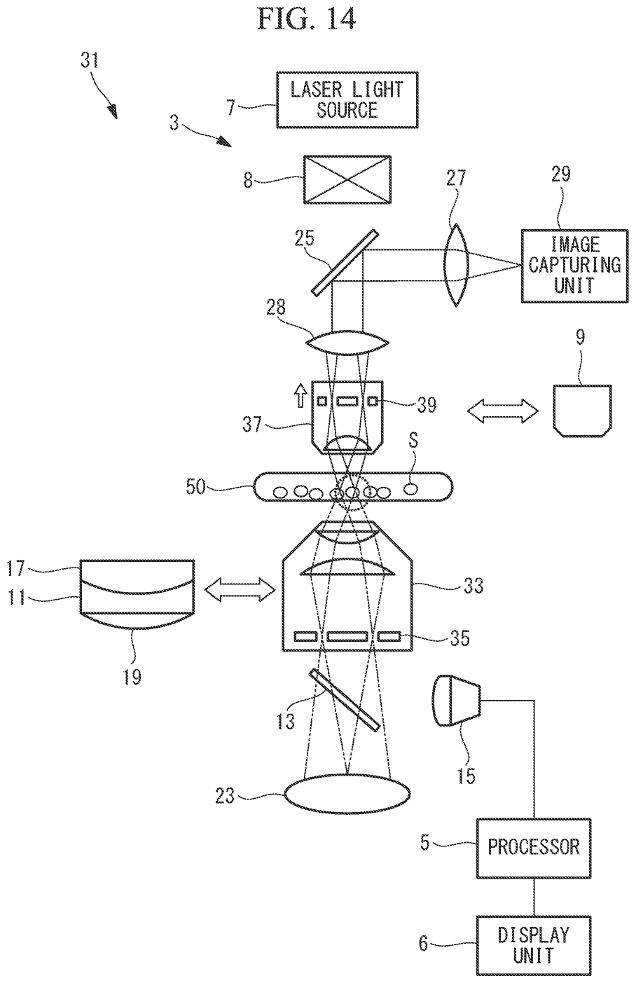

As shown in FIG. 14, an erythrocyte differentiation monitoring apparatus 31 according to this embodiment is different from that in the second embodiment in that phase-difference observation is performed in place of oblique-illumination observation.

In the following description, sections identical to those in the erythrocyte differentiation monitoring apparatus 21 and the erythrocyte differentiation monitoring method according to the second embodiment will be given the same reference signs, and descriptions thereof will be omitted.

The erythrocyte differentiation monitoring apparatus 31 includes a phase-difference condenser lens (light-collecting optical system) 33 that radiates illumination light emitted from the illumination light source 23 onto a cell S within the culture container 50, and also includes a phase-difference objective lens (light-collecting optical system) 37 that collects observation light from the cell S irradiated with the illumination light.

The phase-difference condenser lens 33 has a ring slit 35 therein. Of the illumination light emitted from the illumination light source 23, the ring slit 35 only allows light entering the ring slit 35 to pass therethrough, and blocks light incident on locations other than the ring slit 35.

The phase-difference condenser lens 33 is disposed on a turret (not shown) together with the set of the transmission member 17, the acoustic lens 11, and the correction lens 19. By using the turret, the phase-difference condenser lens 33 and the set of the transmission member 17, the acoustic lens 11, and the correction lens 19 can be selectively disposed on the optical path of the illumination light.

The phase-difference objective lens 37 has a phase plate 39 therein. The phase-difference objective lens 37 is disposed on a turret (not shown) together with the objective lens 9. By using the turret, the phase-difference objective lens 37 and the objective lens 9 can be selectively disposed on the optical path of the illumination light. The phase plate 39 is disposed at a conjugate position with respect to the ring slit 35 of the phase-difference condenser lens 33.

The operation of the erythrocyte differentiation monitoring apparatus 31 having the above-described configuration and the erythrocyte differentiation monitoring method will now be described.

In a case where a cell S is to be monitored by using the erythrocyte differentiation monitoring apparatus 31 and the erythrocyte differentiation monitoring method according to this embodiment, illumination light is first generated from the illumination light source 23 in a state where the phase-difference condenser lens 33 is disposed on the optical path of the illumination light from the illumination light source 23 and the phase-difference objective lens 37 is disposed on the optical path of observation light from the cell S.

After the illumination light emitted from the illumination light source 23 is transmitted through the acoustic-wave reflection member 13, only the illumination light transmitted through the ring slit 35 by the phase-difference condenser lens 33 is radiated onto the cell S. After the observation light emitted from the cell S as a result of the cell S being irradiated with the illumination light is collected by the phase-difference objective lens 37, only the observation light transmitted through the phase plate 39 is reflected by the dichroic mirror 25, and an image of the observation light is captured by the image capturing unit 29. Accordingly, the processor 5 acquires a phase-difference cell image of the cell S.

Subsequently, the phase-difference condenser lens 33 is switched to the set of the transmission member 17, the acoustic lens 11, and the correction lens 19, and the phase-difference objective lens 37 is switched to the objective lens 9. Then, a laser beam in the hemoglobin absorption wavelength range is generated from the laser light source 7. The laser beam emitted from the laser light source 7 is radiated onto the cell S via the scanning unit 8, the dichroic mirror 25, and the objective lens 9.

A photoacoustic wave emitted from the cell S as a result of the cell S being irradiated with the laser beam travels through the transmission member 17, the acoustic lens 11, and the correction lens 19, and is subsequently reflected by the acoustic-wave reflection member 13 and received by the probe 15. Accordingly, the processor 5 acquires a photoacoustic image of the cell S.

Subsequently, the processor 5 superimposes the phase-difference cell image and photoacoustic image of the cell S on each other. Then, the processor 5 executes step SB4 to step SB10 in the flowchart in FIG. 12 so as to determine the state of the cell S.

According to this embodiment, the state of the cell S can be determined by acquiring a high-resolution high-contrast cell image in accordance with phase-difference observation.

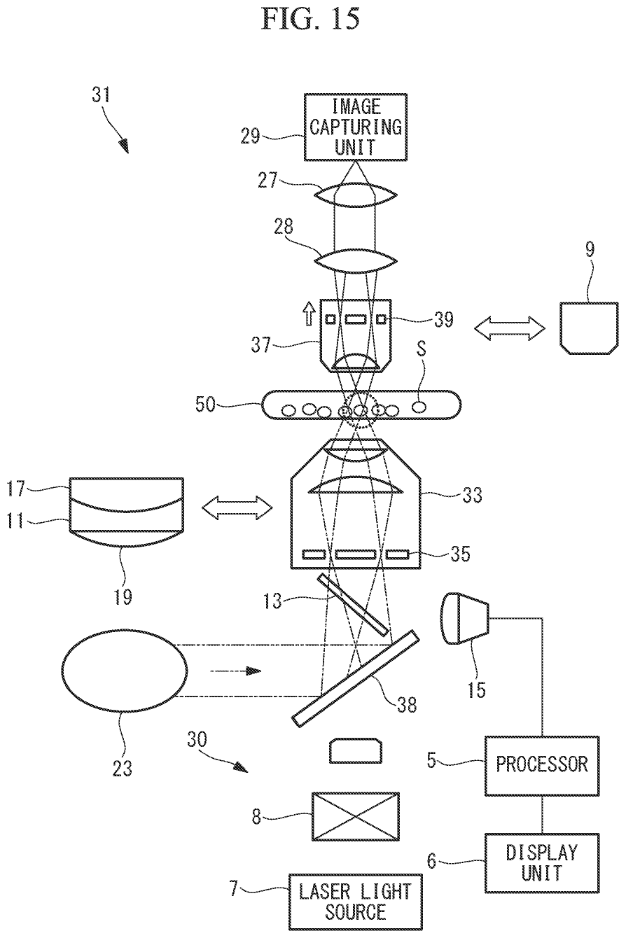

This embodiment described above relates to a case where the erythrocyte differentiation monitoring apparatus 31 includes the photoacoustic optical system 3. Alternatively, for example, the erythrocyte differentiation monitoring apparatus 31 may include the photoacoustic optical system 30, as shown in FIG. 15.

In this case, a turret may be used to switch between the phase-difference condenser lens 33 and the set of the transmission member 17, the acoustic lens 11, and the correction lens 19, and also to switch between the phase-difference objective lens 37 and the objective lens 9. With this configuration, advantages similar to those in this embodiment can be achieved. In FIG. 15, reference sign 38 denotes a mirror that reflects illumination light from the illumination light source 23 toward the phase-difference condenser lens 33 or the set of the transmission member 17, the acoustic lens 11, and the correction lens 19.

As an alternative to the second and third embodiments in which oblique-illumination observation and phase-difference observation are described as examples of an observation method for acquiring a cell image, other observation methods, such as differential-interference observation, may be used. Although information about a cell S prior to differentiating into an erythrocyte is not acquirable since a photoacoustic wave occurs in accordance with the production of hemoglobin, an image can be acquired even for a transparent and colorless cell S prior to differentiating into an erythrocyte by using oblique-illumination observation and phase-difference observation.

### Fourth Embodiment

Next, an erythrocyte differentiation monitoring apparatus and an erythrocyte differentiation monitoring method according to a fourth embodiment of the present invention will be described.

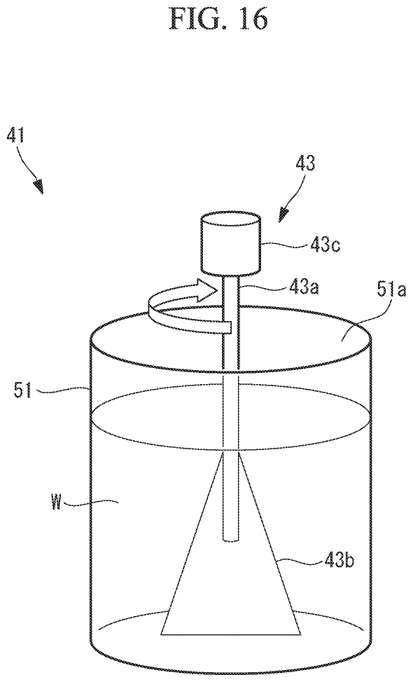

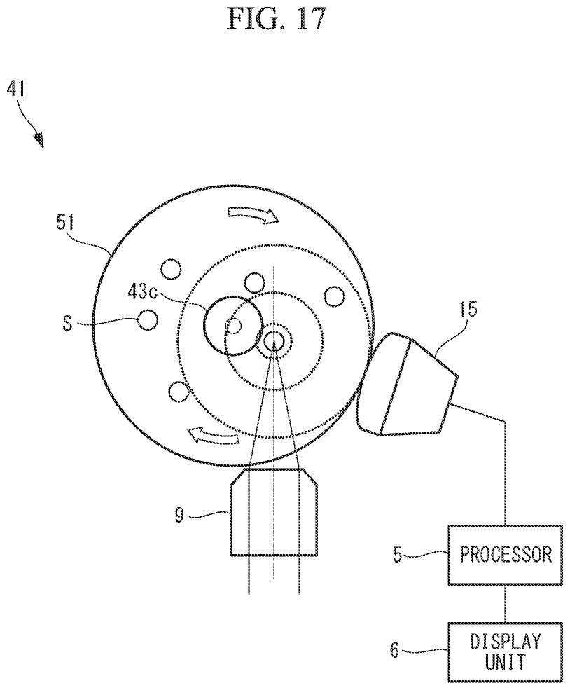

As shown in FIGS. 16 and 17, an erythrocyte differentiation monitoring apparatus 41 according to this embodiment is different from that in the first embodiment in that the erythrocyte differentiation monitoring apparatus 41 is used in a suspension culture process instead of a plane culture process.

In the following description, sections identical to those in the erythrocyte differentiation monitoring apparatus 1 and the erythrocyte differentiation monitoring method according to the first embodiment will be given the same reference signs, and descriptions thereof will be omitted.

The erythrocyte differentiation monitoring apparatus 41 uses a culture container (container) 51, such as a bioreactor, for suspension-culturing an erythrocyte. The culture container 51, such as a bioreactor, used in this embodiment has a closed-end cylindrical shape whose upper surface 51a is blocked. The culture container 51 is composed of an optically-transparent material. A suspension culture process using, for example, a bioreactor is advantageous in that a large number of cells S can be cultured at once, as compared with a plane culture process using, for example, a flask or a dish.

The erythrocyte differentiation monitoring apparatus 41 includes an agitating mechanism 43 that agitates a culture solution (culture medium) W within the culture container 51. The agitating mechanism 43 includes a shaft 43a to be inserted into the culture container 51 via the upper surface 51a of the culture container 51, an agitating blade 43b provided on the shaft 43a, and a motor 43c that rotates the shaft 43a about its longitudinal axis. The agitating mechanism 43 agitates the culture solution W within the culture container 51 so that the cells S float substantially uniformly in the culture solution W in a distributed manner. Accordingly, it is possible to assume that the cells S exist uniformly within the culture container 51, so that the dependency on the measurement position in, for example, photoacoustic wave measurement and cell density measurement, to be described later, decreases.

The erythrocyte differentiation monitoring apparatus 41 does not include the scanning unit 8, and uses the objective lens 9 to focus a laser beam emitted from the laser light source 7, thereby radiating the laser beam onto a specific position in the culture solution W. In other words, the laser beam is not scanned, and the irradiation position of the laser beam is fixed.

The probe 15 receives a photoacoustic wave emitted from each cell S and traveling through the irradiation position of the laser beam in the culture solution W. A liquid photoacoustic transmission medium, such as water, transmitting a photoacoustic wave fills the space between the probe 15 and the side surface of the culture container 51.

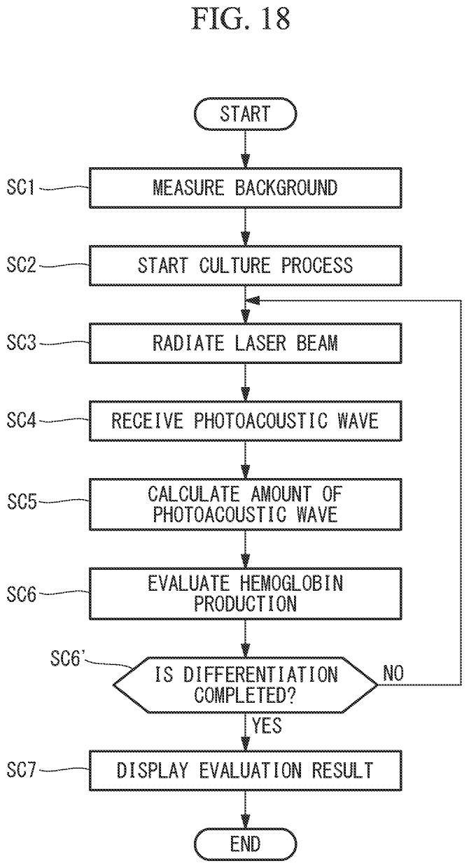

The operation of the erythrocyte differentiation monitoring apparatus 41 having the above-described configuration and the erythrocyte differentiation monitoring method will be described below with reference to a flowchart in FIG. 18.

In a case where a cell S is to be monitored by using the erythrocyte differentiation monitoring apparatus 41 according to this embodiment, a laser beam in the hemoglobin absorption wavelength range is first radiated onto the culture solution W within the culture container 51 before the cell S is accommodated in the culture container 51. Then, a photoacoustic signal emitted from the medium component and other components is acquired by the probe 15. The photoacoustic signal acquired by the probe 15 is transmitted as a background signal to the processor 5 (step SC1).

Subsequently, the culture process of the cell S starts while the agitating mechanism 43 agitates the culture solution W within the culture container 51 (step SC2). Then, in the state where the culture solution W is agitated, the laser light source 7 generates a laser beam in the hemoglobin absorption wavelength range. The laser beam emitted from the laser light source 7 is radiated onto a specific position within the culture container 51 via the objective lens 9 (step SC3).

When the cell S floating in the culture solution W passes through the irradiation position of the laser beam, if the cell S has differentiated into an orthochromatic erythroblast, the laser beam is absorbed by hemoglobin, so that a photoacoustic wave is emitted from the cell S. The photoacoustic wave emitted from the cell S is received by the probe 15 (step SC4).

Subsequently, the processor 5 calculates the intensity of the photoacoustic wave from the cell S by subtracting the background signal from the photoacoustic signal transmitted from the probe 15 (step SC5). Then, the processor 5 evaluates the progress of differentiation of the cell S into an erythrocyte based on the intensity of the photoacoustic wave from the cell S (step SA6).

The laser beam is continuously radiated from the laser light source 7 to the specific position in the culture solution W, or is radiated from the laser light source 7 to the specific position in the culture solution W at certain time intervals, such as every hour, so that a temporal change in the intensity of the photoacoustic wave received by the probe 15 is monitored by the processor 5. Then, the processor 5 determines whether or not the differentiation of the cell S is completed (step SC6′).

As the differentiation of the cell S within the culture container 51 progresses, the amount of hemoglobin in the cell S increases. Thus, the intensity of the photoacoustic wave received by the probe 15 also increases over time. When the intensity of the photoacoustic wave received by the probe 15 no longer increases, it is determined that the differentiation of the cell S is completed (“YES” in step SC6′). Then, the culture process is terminated, and the cell S is extracted. It may be determined that the differentiation is completed when the detected intensity of the photoacoustic wave exceeds a predetermined threshold value. The evaluation result obtained by the processor 5, the calculated amount of produced hemoglobin, and the estimated cell type are individually displayed immediately by the display unit 6 or are collectively displayed by the display unit 6 (step SC7).

As described above, in the erythrocyte differentiation monitoring apparatus 41 and the erythrocyte differentiation monitoring method according to this embodiment, the degree of progress with respect to differentiation of a plurality of cells S contained within the culture container 51 can be ascertained. Moreover, by recording a temporal change in the intensity of a photoacoustic wave during a culture process, it is possible to make comparisons and predictions with respect to the progress of the culture process by comparing the photoacoustic wave with a photoacoustic wave when another subsequent culture process is performed.

Since the culture solution W and differentiated cells S are colored, the optical transparency in the culture container 51 is poor in a suspension culture process. Thus, there are limitations in that it is difficult to perform observation using optical methods, such as image observation and absorbance observation. In addition, the use of optical measurement in a suspension culture process causes light scattering and light reflection to occur, leading to an increase in noise and attenuation of a target signal. Photoacoustic measurement involves detecting an acoustic wave that is not affected by light scattering and light reflection. Furthermore, the culture solution W being a liquid has high acoustic-wave propagation efficiency, so that a target signal can be genuinely acquired. Moreover, when a laser beam is radiated onto a cell S, a photoacoustic wave is emitted evenly from all directions from the cell S, specifically, for example, spherically, that is, radially, from the center of the cell S. Therefore, the detection position of a photoacoustic wave may be located anywhere. This is advantageous in that the degree of freedom in the measurement position is higher than in optical measurement, such as image observation or absorbance observation.

The total number of the plurality of cells S within the culture container 51 may change due to, for example, division occurring between the start and the end of the culture process. In this embodiment, the total photoacoustic wave level is ascertainable, whereas information about the number of cells S is unknown. Thus, for example, by using the cell density in the culture solution W, a change in the intensity of a photoacoustic wave per cell in a period from a stem cell to an erythrocyte, that is, the differentiation efficiency of each cell S, may be determined.

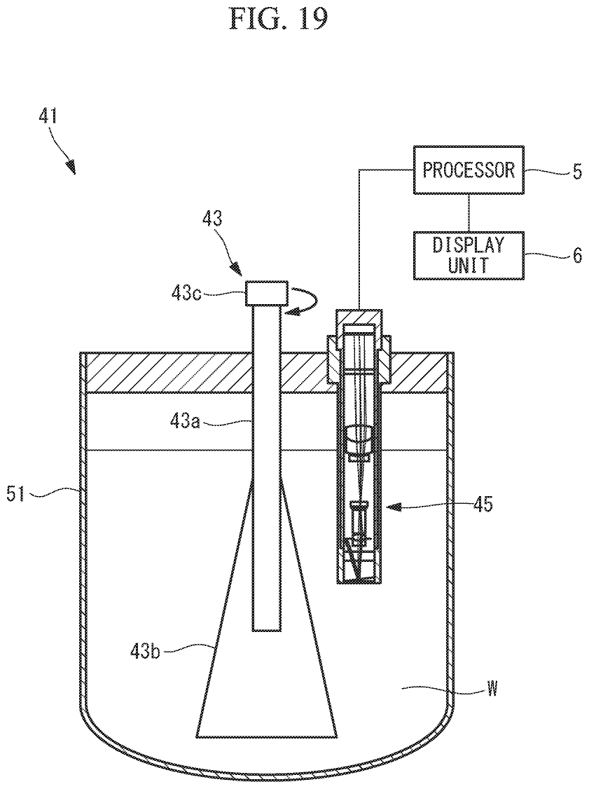

If the cell density (cells/mm³) in the culture solution W is to be calculated, for example, a stereo measurement device (measuring unit) 45 shown in FIG. 19 may be used. The stereo measurement device 45 is inserted into the culture solution W in the culture container 51 to perform stereo measurement.

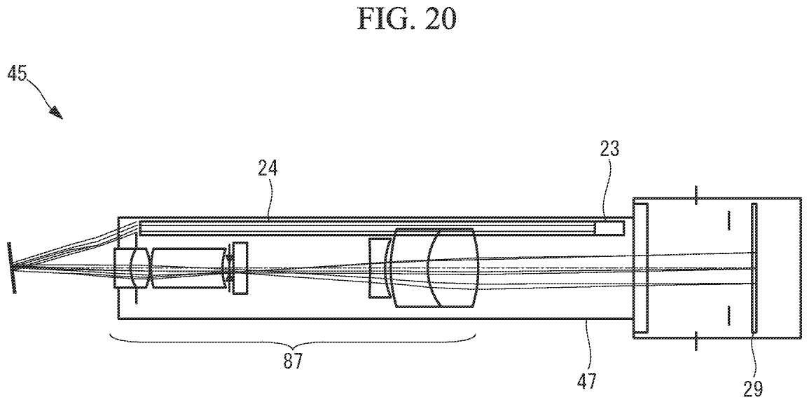

As shown in FIG. 20, the stereo measurement device 45 includes an illumination light source 23, a stereo optical system 47 that forms two parallax images as viewed from different viewpoints with respect to the same cell S floating in the culture solution W, an image capturing unit 29 that captures the two images formed by the stereo optical system 47, and an optically-transparent housing 49 that accommodates the illumination light source 23, the stereo optical system 47, and the image capturing unit 29. In FIG. 20, reference sign 24 denotes a light guide fiber that optically guides illumination light emitted from the illumination light source 23.

Furthermore, the processor 5 may determine a temporal change in the intensity of a photoacoustic wave per cell by using the following method. For example, the processor 5 determines the positions of cells S included in each of the two images acquired by the image capturing unit 29, and calculates the cell density in the culture solution W based on the number of cells S existing within a predetermined region.

When the cell density in the culture solution W is calculated, the processor 5 determines the volume at a focal point of a laser beam by using a beam waist value corresponding to a minimum beam diameter of the laser beam, and calculates the photoacoustic-wave intensity per unit volume (photoacoustic-wave intensity/mm³).

Subsequently, the processor 5 divides the calculated photoacoustic-wave intensity per unit volume (photoacoustic-wave intensity/mm³) by the cell density (cells/mm³) in the culture solution W. Accordingly, the photoacoustic-wave intensity per cell (photoacoustic-wave intensity/cell) at the measurement time point is calculated. When the photoacoustic-wave intensity per unit volume no longer increases, it is determined that the differentiation of each cell S is completed, and the photoacoustic-wave intensity at the time of completion of the differentiation is stored. It may be determined that the differentiation is completed when the detected photoacoustic-wave intensity exceeds a predetermined threshold value.

With this configuration, it is possible to determine the absolute intensity and change of a photoacoustic wave per cell with the progress of differentiation under the conditions of an apparatus and container used by the user. Moreover, by performing a comparison with the photoacoustic-wave intensity per cell obtained in a past experiment, it is possible to perform a comparison with respect to the overall differentiation efficiency of the plurality of cells S. By performing the comparison with respect to the differentiation efficiency, if the efficiency is low, the user can take countermeasures, such as stopping the culture process in mid-course or reviewing the culture technique, thereby ultimately increasing the culture efficiency.

As an alternative to this embodiment in which the culture container 51 composed of an optically-transparent material and having a closed-end cylindrical shape is described as an example of the container, the container may have a freely-chosen shape, such as a bag-like shape, a spherical shape, or a box-like shape. For example, a disposable bag-like culture container (culture bag) may be used. Furthermore, the container used may be composed of a freely-chosen material, such as a rigid material or a soft material like vinyl. Moreover, the container does not have to be entirely transparent and may partially have a transparent section that transmits a laser beam. Since a photoacoustic wave is generated so long as a laser beam is radiated onto a cell S, the material and shape of the container are not particularly limited so long as the container partially has a transparent section. If the container is composed of a soft material, such a container is advantageous in that the probe 15 can be readily brought into close contact therewith when the probe 15 is set outside the container.

As mentioned above, the application of a photoacoustic technique to a suspension culture process is advantageous in terms of the degree of freedom in the measurement position. As an alternative to this embodiment in which the probe 15 is provided outside the culture container 51, the probe 15 may be provided inside the culture container 51.

For example, the probe 15 may be simply disposed inside the culture container 51, or the probe 15 may be laminar and be bonded within the culture container 51. Alternatively, the probe 15 may have a planar shape. In this case, the probe 15 may be bonded to the base or the lid of the culture container 51. As another alternative, the probe 15 may have a rounded shape. In this case, the probe 15 may be bonded to the side surface of the culture container 51.

According to these modifications, a photoacoustic wave emitted spherically from the center of a cell S, that is, from all directions from the cell S, can be uniformly received by the probe 15. Moreover, since the probe 15 is not provided outside the culture container 51, the entire system can be reduced in size.

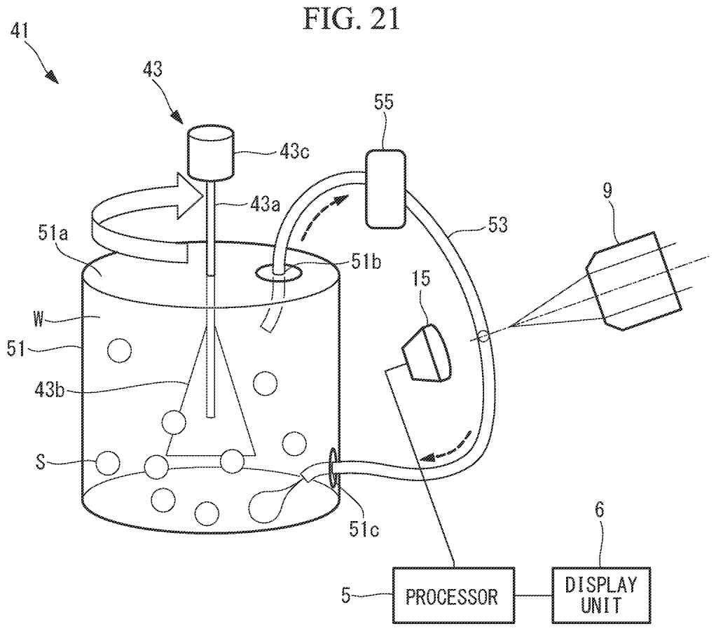

Although the above-described embodiment relates to an example where a photoacoustic wave is received both inside and outside the culture container 51, an additional flow path may be provided outside the culture container 51 to form a differentiation monitoring location. For example, as shown in FIG. 21, a suction port 51b is provided in the upper surface of the culture container 51, such as a bioreactor and a culture bag, and a return port 51c is provided in the sidewall of the culture container 51. A tubular member 53 serving as a flow path that connects the suction port 51b and the return port 51c is set outside the culture container 51.

The laser light source 7 radiates a laser beam onto a section of the tubular member 53. Then, a photoacoustic wave emitted from a cell S and traveling through the laser-beam irradiated section in the tubular member 53 may be received by the probe 15. An additional observation location for retaining the culture solution W and the cells S may be provided at an intermediate location of the tubular member 53.

A liquid feed pump 55 may be disposed at a section of the tubular member 53 to generate a liquid current. The liquid feed pump 55 used may be, for example, a diaphragm pump. The liquid feed pump 55 may be switched between on and off modes in accordance with a drive signal transmitted from the processor 5. The liquid feed pump 55 may be constantly in the on mode, or may be set to the on mode only when a photoacoustic wave is to be desirably acquired. It is desirable that the liquid feed pump 55 have no risk of breaking or damaging the cells S delivered through the tubular member 53.

The tubular member 53 used may be, for example, a soft tube composed of silicon or rubber or a rigid tube composed of metal. The material of the tubular member 53 is not particularly limited so long as at least a section of the tubular member 53 has acoustic-wave propagation properties. The tubular member 53 may at least partially have a hole that can be used for laser-beam irradiation. The hole used for the laser-beam irradiation is not necessary if the tubular member 53 has high optical transparency.

If the probe 15 is to be set outside the culture container 51, the probe 15 may be set anywhere so long as the probe 15 is in close contact with the tubular member 53. For example, the probe 15 may be disposed at the same side as the laser-beam irradiation position relative to the tubular member 53, that is, at the side with the laser light source 7 (reflective type). Alternatively, the probe 15 may be disposed at the opposite side from the laser-beam irradiation position, that is, at the side opposite from the laser light source 7 (transmissive type). As another alternative, the probe 15 may be laminar. In this case, the probe 15 may be bonded inside the tubular member 53.

In these modifications, a photoacoustic wave is acquired by providing another flow path in addition to the culture container 51. In this configuration, the distance that the light and photoacoustic wave travel is shorter than that in the configuration where a laser beam is directly radiated into the culture container 51. Accordingly, the light and photoacoustic wave are less attenuated, so that noise is less likely to occur therein, thereby enabling highly-accurate observation. Moreover, by increasing the length of the tubular member 53, the degree of freedom in the observation position becomes higher, thereby improving the workability.

As an alternative to each of the above embodiments in which a laser beam with a wavelength of 555 nm as a peak absorption wavelength for hemoglobin (Hb) is radiated, a laser beam in another hemoglobin absorption wavelength range may be used. For example, if phenol red is to be used as a culture medium, a laser beam with a wavelength near 1000 nm as a near-infrared wavelength not belonging to the phenol-red absorption wavelength range may be used.

Furthermore, for example, if the oxygen partial pressure is high, there is a possibility of a mixture of oxygenated hemoglobin (HbO₂). In that case, laser beams with wavelengths of 541 nm and 576 nm as peak absorption wavelengths for oxygenated hemoglobin (HbO₂) may be radiated. Furthermore, by using a plurality of wavelengths, the mixture ratio of hemoglobin (Hb) and oxygenated hemoglobin (HbO₂) may be determined.

Before a laser beam reaches a desired cell S, the laser beam may possibly be absorbed by another cell S existing in front of the desired cell S or by a culture medium, such as phenol red, thus causing the probe 15 to receive a photoacoustic wave other than a photoacoustic wave from the desired cell S. In order to prevent this, a two-photon excitation photoacoustic technique may be used.

A two-photon excitation photoacoustic technique involves, for example, focusing and radiating a pulsed laser beam with high peak power and a pulse width of several hundreds of femtoseconds onto a cell S. When a laser beam with a spatially and temporally high photon density is radiated by focusing a laser beam with high peak power onto a single spot, if the energy difference between the ground state and the excited state of a molecule becomes substantially twice the photon energy, two photons are simultaneously absorbed, and the molecule transitions to the excited state, thus causing two-photon excitation to occur. With the two-photon excitation photoacoustic technique, absorption occurs selectively only in a micro-region near the focal point where the photon density is high, thereby enabling high-contrast observation without a photoacoustic wave being generated from, for example, a cell S other than the cell S to be desirably observed or phenol red serving as a medium component due to laser beams converging at the focal point.

In the case where the two-photon excitation photoacoustic technique is to be used, for example, a wavelength that is close to twice the wavelength of 555 nm as a peak absorption wavelength for hemoglobin (Hb) or a wavelength that is close to twice the wavelengths of 541 nm and 576 nm as peak absorption wavelengths for oxygenated hemoglobin (HbO₂) may be used. Since half of the wavelength that is close to twice the wavelength of 555 nm and half of the wavelength that is close to twice the wavelengths of 541 nm and 576 nm are both hemoglobin absorption wavelengths, two-photon excitation occurs.

Furthermore, since these wavelengths are near-infrared wavelengths from near 1000 nm to near 1150 nm, the wavelengths are not absorbed by phenol red serving as a culture medium, so that a photoacoustic wave based on two-photon excitation can be efficiently generated. Since a near-infrared wavelength near 1000 nm is outside the hemoglobin absorption wavelength as well as the absorption wavelength range of phenol red serving as a medium component, the intensity of an input laser beam is not absorbed by the culture medium, so that the photoacoustic-wave generation efficiency with respect to the intensity of the input laser beam may be ensured.

Although the embodiments of the present invention have been described above in detail with reference to the drawings, specific configurations are not limited to these embodiments, and design modifications are also to be included so long as they do not depart from the scope of the present invention. For example, the present invention is not limited to being applied to the above embodiments and modifications, and may be applied to an embodiment obtained by appropriately combining these embodiments and modifications; hence, the present invention is not particularly limited.

The following aspects can be derived from this embodiment.

A first aspect of the present invention provides an erythrocyte differentiation monitoring apparatus including a laser light source that radiates a laser beam in a hemoglobin absorption wavelength range onto a cell within a culture container, an acoustic wave receiver that receives a photoacoustic wave emitted from the cell as a result of the cell being irradiated with the laser beam, and a processor that evaluates a progress of differentiation of the cell into an erythrocyte based on an intensity of the photoacoustic wave received by the acoustic wave receiver and outputs an evaluation result.

In the process of differentiation from a stem cell into an erythrocyte, the production of hemoglobin starts when the stem cell differentiates into an orthochromatic erythroblast. According to this aspect, when the laser light source radiates a laser beam in the hemoglobin absorption wavelength range onto a cell, if the cell has differentiated into an orthochromatic erythroblast, a photoacoustic wave is emitted from the hemoglobin in the cell, and the acoustic wave receiver receives the photoacoustic wave. In contrast, if the cell has not properly differentiated, hemoglobin is not produced in the cell, so that a photoacoustic wave is not emitted.

Therefore, the processor can determine whether or not the differentiation into an erythrocyte is properly progressing based on the intensity of the photoacoustic wave received by the acoustic wave receiver. Consequently, the differentiation from a stem cell into an erythrocyte can be quantitatively monitored in a simple and accurate manner, as compared with a case where the differentiation of the cell is confirmed visually.

In the erythrocyte differentiation monitoring apparatus according to the above aspect, the processor may calculate an amount of hemoglobin produced in the cell based on the intensity of the photoacoustic wave received by the acoustic wave receiver.

By radiating a laser beam in the hemoglobin absorption wavelength range, the amount of absorption of the laser beam and the intensity of the photoacoustic wave emitted from the cell increase with an increasing amount of hemoglobin in the cell. In other words, the amount of hemoglobin produced in the cell is proportional to the intensity of the photoacoustic wave received by the acoustic wave receiver. Therefore, with the above configuration, the amount of hemoglobin produced in the cell can be readily calculated.

In the erythrocyte differentiation monitoring apparatus according to the above aspect, the processor may estimate a cell type of the cell from the calculated amount of produced hemoglobin.

In the process of differentiation from a stem cell into an erythrocyte, the amount of produced hemoglobin increases gradually from an orthochromatic erythroblast to a reticulocyte, whereas the amount of produced hemoglobin increases rapidly from a reticulocyte to an erythrocyte. Therefore, with the above configuration, the cell type can be readily estimated.

In the erythrocyte differentiation monitoring apparatus according to the above aspect, if the processor determines that the differentiation is not completed, the processor may cause the laser light source to radiate the laser beam onto the cell again, and acquire a temporal change in the intensity of the photoacoustic wave.

With this configuration, a stem cell can be monitored until it differentiates into an erythrocyte.

In the erythrocyte differentiation monitoring apparatus according to the above aspect, the processor may evaluate completion of the differentiation based on a threshold value for the intensity of the photoacoustic wave or a rate of the temporal change in the intensity of the photoacoustic wave.

The erythrocyte differentiation monitoring apparatus according to the above aspect may further include a display unit that displays the temporal change.

With this configuration, the temporal change in the photoacoustic wave emitted from the cell can be readily ascertained from the display unit.

In the erythrocyte differentiation monitoring apparatus according to the above aspect, the processor may generate a photoacoustic image based on a positional coordinate from where the photoacoustic wave is emitted and the intensity of the photoacoustic wave.

With the above configuration, the position of the cell that has properly differentiated from a stem cell into an erythrocyte within the container can be visually identified from the generated photoacoustic image.

The erythrocyte differentiation monitoring apparatus according to the above aspect may further include a display unit that displays an image. The processor may display a superimposed image on the display unit. The superimposed image is obtained by superimposing the photoacoustic image and a cell image on each other. The cell image is obtained by capturing an image of observation light from the cell.

The shape of the cell can be determined from the cell image obtained by capturing the image of the observation light from the cell. Therefore, with the above configuration, the position and the shape of the cell that has properly differentiated from a stem cell into an erythrocyte within the container can both be visually identified from the superimposed image of the photoacoustic image and the cell image displayed on the display unit.

In the erythrocyte differentiation monitoring apparatus according to the above aspect, based on the photoacoustic image and the cell image of a plurality of cells included in a specific region within the culture container, the processor may calculate a percentage of cells differentiated into erythrocytes among the plurality of cells included in the specific region.

The erythrocyte differentiation monitoring apparatus according to the above aspect may further include an illumination light source that radiates illumination light onto the cell, and an image capturing unit that captures the image of the observation light emitted from the cell as a result of the cell being irradiated with the illumination light. The processor may generate the cell image based on image information of the cell acquired by the image capturing unit.

With the above configuration, the photoacoustic image and the cell image of the cell can both be acquired. Therefore, it is not necessary to move the container between a case where a photoacoustic image of the same cell is to be acquired and a case where a cell image thereof is to be acquired. Thus, the same cell can be readily and accurately associated between the photoacoustic image and the cell image.

In the erythrocyte differentiation monitoring apparatus according to the above aspect, the illumination light source may obliquely illuminate the cell from a direction inclined relative to an optical axis of the image capturing unit.

With the above configuration, a three-dimensional cell image of a transparent and colorless cell can be acquired.

The erythrocyte differentiation monitoring apparatus according to the above aspect may further include a light-collecting optical system for phase-difference observation.

With the light-collecting optical system for phase-difference observation, a high-resolution high-contrast cell image of the cell can be acquired.

In the erythrocyte differentiation monitoring apparatus according to the above aspect, the laser beam may be a light beam with a near-infrared wavelength.

A near-infrared wavelength is in the hemoglobin absorption wavelength range but is not in the phenol-red absorption wavelength range. Therefore, with the above configuration, if phenol red is used as a culture medium, absorption of the laser beam by the phenol red can be prevented.

In the erythrocyte differentiation monitoring apparatus according to the above aspect, the culture container may be a bioreactor or a culture bag.

In the erythrocyte differentiation monitoring apparatus according to the above aspect, the culture container may be the bioreactor, and the bioreactor may be equipped with an agitating blade.

With this configuration, the differentiation of the cell undergoing a suspension culture process can be monitored.

The erythrocyte differentiation monitoring apparatus according to the above aspect may further include a measuring unit that measures a cell density in a culture solution within the culture container.

With this configuration, the cell density in the culture solution measured by the measuring unit can be used to determine a change in the intensity of the photoacoustic wave per cell in a period from a stem cell to an erythrocyte, that is, the differentiation efficiency of each cell.

In the erythrocyte differentiation monitoring apparatus according to the above aspect, the acoustic wave receiver may be laminar and be set inside the culture container.

With this configuration, since the acoustic wave receiver is not disposed outside the culture container, the entire system can be reduced in size.

The erythrocyte differentiation monitoring apparatus according to the above aspect may further include a tubular member through which the cell within the culture container can travel together with a culture solution. The tubular member may have opposite longitudinal ends that are connected to the culture container and may be disposed outside the culture container. The laser light source may radiate the laser beam onto the cell traveling through the tubular member.

In this configuration, the narrow tubular member is used so that the distance that the light and the photoacoustic wave travel is shorter than that in a case where the laser beam is radiated onto the cell within the culture container. Accordingly, attenuation of the light and the photoacoustic wave is suppressed, and noise is reduced, thereby enabling highly-accurate observation.

A second aspect of the present invention provides an erythrocyte differentiation monitoring method including radiating a laser beam in a hemoglobin absorption wavelength range onto a cell, receiving a photoacoustic wave emitted from the cell as a result of the cell being irradiated with the laser beam, and evaluating a progress of differentiation of the cell into an erythrocyte based on an intensity of the received photoacoustic wave and outputting an evaluation result.

The erythrocyte differentiation monitoring method according to the above aspect may further include radiating the laser beam onto the cell again if it is determined by the evaluation result that the differentiation is not completed, and acquiring a temporal change in the intensity of the photoacoustic wave.

### REFERENCE SIGNS LIST

1, 21, 31, 41 erythrocyte differentiation monitoring apparatus
5 processor
6 display unit
7 laser light source
15 probe (acoustic wave receiver)
23 illumination light source
29 image capturing unit
33 phase-difference condenser lens (light-collecting optical system)
37 phase-difference objective lens (light-collecting optical system)
45 stereo measurement device (measuring unit)
50, 51 culture container (container)
53 tubular member
S cell

1. An erythrocyte differentiation monitoring apparatus comprising:
a laser light source that radiates a laser beam in a hemoglobin absorption wavelength range onto a cell within a culture container;
an acoustic wave receiver that receives a photoacoustic wave emitted from the cell as a result of the cell being irradiated with the laser beam; and
a processor that evaluates a progress of differentiation of the cell into an erythrocyte based on an intensity of the photoacoustic wave received by the acoustic wave receiver and outputs an evaluation result.

2. The erythrocyte differentiation monitoring apparatus according to claim 1, wherein the processor calculates an amount of hemoglobin produced in the cell based on the intensity of the photoacoustic wave received by the acoustic wave receiver.

3. The erythrocyte differentiation monitoring apparatus according to claim 2, wherein the processor estimates a cell type of the cell from the calculated amount of produced hemoglobin.

4. The erythrocyte differentiation monitoring apparatus according to claim 1, wherein if the processor determines that the differentiation is not completed, the processor causes the laser light source to radiate the laser beam onto the cell again, and acquires a temporal change in the intensity of the photoacoustic wave.

5. The erythrocyte differentiation monitoring apparatus according to claim 4, wherein the processor evaluates completion of the differentiation based on a threshold value for the intensity of the photoacoustic wave or a rate of the temporal change in the intensity of the photoacoustic wave.

6. The erythrocyte differentiation monitoring apparatus according to claim 4, further comprising a monitor that displays the temporal change.

7. The erythrocyte differentiation monitoring apparatus according to claim 1, wherein the processor generates a photoacoustic image based on a positional coordinate from where the photoacoustic wave is emitted and the intensity of the photoacoustic wave.

8. The erythrocyte differentiation monitoring apparatus according to claim 7, further comprising:
a monitor that displays an image,
wherein the processor displays a superimposed image on the monitor, the superimposed image being obtained by superimposing the photoacoustic image and a cell image on each other, the cell image being obtained by capturing an image of observation light from the cell.

9. The erythrocyte differentiation monitoring apparatus according to claim 8, wherein, based on the photoacoustic image and the cell image of a plurality of cells included in a specific region within the culture container, the processor calculates a percentage of cells differentiated into erythrocytes among the plurality of cells included in the specific region.

10. The erythrocyte differentiation monitoring apparatus according to claim 8, further comprising:
an illumination light source that radiates illumination light onto the cell; and
an imager that captures the image of the observation light emitted from the cell as a result of the cell being irradiated with the illumination light,
wherein the processor generates the cell image based on image information of the cell acquired by the imager.

11. The erythrocyte differentiation monitoring apparatus according to claim 10, wherein the illumination light source obliquely illuminates the cell from a direction inclined relative to an optical axis of the imager.

12. The erythrocyte differentiation monitoring apparatus according to claim 10, further comprising a light-collecting optical system that comprises a lens for phase-difference observation.

13. The erythrocyte differentiation monitoring apparatus according to claim 1, wherein the laser beam is a light beam with a near-infrared wavelength.

14. The erythrocyte differentiation monitoring apparatus according to claim 1, wherein the culture container is a bioreactor or a culture bag.

15. The erythrocyte differentiation monitoring apparatus according to claim 14, further comprising a measure that measures a cell density in a culture solution within the culture container.

16. The erythrocyte differentiation monitoring apparatus according to claim 14, wherein the acoustic wave receiver is laminar and is set inside the culture container.

17. The erythrocyte differentiation monitoring apparatus according to claim 14, further comprising:
a tube through which the cell within the culture container can travel together with a culture solution,
wherein the tube has opposite longitudinal ends that are connected to the culture container and is disposed outside the culture container, and
wherein the laser light source radiates the laser beam onto the cell traveling through the tube.

18. The erythrocyte differentiation monitoring apparatus according to claim 1, wherein the photoacoustic wave received by the acoustic wave receiver includes a photoacoustic wave emitted from the cell which is still undergoing a differentiation process into the erythrocyte.

19. An erythrocyte differentiation monitoring method comprising:
radiating a laser beam in a hemoglobin absorption wavelength range onto a cell;
receiving a photoacoustic wave emitted from the cell as a result of the cell being irradiated with the laser beam; and
evaluating a progress of differentiation of the cell into an erythrocyte based on an intensity of the received photoacoustic wave and outputting an evaluation result.

20. The erythrocyte differentiation monitoring method according to claim 19, further comprising:
radiating the laser beam onto the cell again if it is determined by the evaluation result that the differentiation is not completed; and
acquiring a temporal change in the intensity of the photoacoustic wave.
